(19) United States
(12) Patent Application Publication
(10) Pub. No.: US 2007/0225657 A1
(43) Pub. Date: Sep. 27, 2007

(54) Dosing Device

(76) Inventor: Edgar Hommann, Grossaffoltern (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/559,967

(22) Filed: Nov. 15, 2006

Related U.S. Application Data
(63) Continuation of application No. PCT/CH05/00276, filed on May 18, 2005.

(30) Foreign Application Priority Data
May 25, 2004 (DE) 10 2004 025 546.6

(51) Int. Cl. A61M 5/00

(52) U.S. Cl. 604/207

(57) ABSTRACT

A dosing device for an administering apparatus, such as an injection device, for administering a substance in doses including a dosage setting mechanism for setting a dosage of the substance to be administered by a first rotational movement, and a scale which can be rotated by a second rotational movement for reading or displaying the dosage set, wherein a gear couples the first rotational movement to the second rotational movement such that the second rotational movement is slower then the first rotational movement.

### CROSS-REFERENCE TO RELATED APPLICATION(S)

This application claims priority to International Patent Application No. PCT/CH2005/000276, filed on May 18, 2005, which claims priority to German Application No. 10 2004 025 546.6, filed on May 25, 2004, the contents of both of which are incorporated herein by reference.

### BACKGROUND

The present invention relates to devices for administrating, delivering, dispensing, injecting or infusing substances, and to methods of making and using such devices. More particularly, it relates to administering apparatus, including injection, infusion or inhalation apparatus, comprising a scale or display.

In many examples of the aforesaid apparatus, it is possible to set or select different sizes of doses or dosages, wherein it is desirable to be able to read, from the apparatus, the dose set or selected. To this end, a scale can be associated with the apparatus.

### SUMMARY

It is an object of the present invention to provide a dosing device, and a device or apparatus for administering or injecting a substance comprising the dosing device, the dosing device comprising a scale which allows a compact design for the device.

In accordance with the present invention, a dosing device is provided for an injection apparatus, to be able to administer a substance in doses. The substance may be, in particular, a medical substance or medicine, in particular in a fluid or liquid state. The medical substance can, for example, be insulin.

In one embodiment, the present invention comprises dosing device or mechanism for an administering apparatus, such as an injection device, for administering a substance in doses, wherein the dosing device comprises a dosage setting mechanism for setting a dosage of the substance to be administered by a first rotational movement, and a scale which can be rotated by a second rotational movement for reading or displaying the dosage set, wherein a gear couples the first rotational movement to the second rotational movement such that the second rotational movement is slower then the first rotational movement.

In one embodiment, the present invention comprises a dosing device for an administering apparatus for administering a substance in doses, the dosing device comprising a dose selector for selecting a dose by a first rotational movement and a scale which can be rotated by a second rotational movement to display the selected dose, wherein the first and second rotational movements are operably coupled or linked such that the second rotational movement is slower then the first rotational movement.

In one embodiment, the dosing device comprises a dosage setting means for setting or selecting a dosage of the substance to be administered. The dosage is set by a first rotational movement which then defines a delivery stroke performed by a drive device. For delivery, the drive device acts on a driven device. The driven device comprises a stroke setting sleeve which acts on a piston of an ampoule to deliver the pre-set dosage. The dosage setting means, for example, comprises an axial rod and the stroke setting sleeve, wherein the first rotational movement rotates the axial rod. This rotational movement is then converted via an outer thread of the axial rod, which engages with an inner thread of the stroke setting sleeve, wherein the stroke setting sleeve is mounted such that it cannot be rotated and thus performs a translational movement in the longitudinal direction when the inner thread is rotated. Shifting the stroke setting sleeve in the longitudinal direction then sets the dosage, since the stroke setting sleeve acts an the piston and travels a pre-set delivery stroke in the longitudinal or axial direction, irrespective of the position from which the stroke setting sleeve starts. In this way, a different amount of fluid is delivered, depending an the position of the stroke setting sleeve in the axial direction, as defined by the dosing device. If the stroke setting sleeve has been moved closer to the piston by the dosing device before the delivery stroke of pre-set length is performed, then more liquid will be delivered than if this has not occurred.

In some embodiments, a dosing device in accordance with the present invention comprises a rotating scale for reading or displaying the dosage set. This scale can be rotated by a second rotational movement. In some embodiments, the second rotational movement is coupled to the first rotational movement, but, in some preferred embodiments, differs in speed from the first rotational movement. For example, the second rotational movement may be slower than the first rotational movement. This means that if a first rotational movement by a first angle is, for example, performed by hand, this causes a second rotational movement by a second angle due to the coupling, said second angle being smaller than said first angle. The reduction is such that the rotational speed of the scale is more than a factor of 2, 4, 6, 8, 10 or 20 smaller than the rotational speed of the first rotational movement with which the dosage is set. In this way, the scale can be configured to be very compact. In some preferred embodiments, it is thus possible to cover an entire range of possible dosing amounts with a single turn of the scale. This allows a particularly compact configuration for the dosing device, since a translational and, therefore, space-consuming movement of the scale to also display larger dosing amounts, is not necessary.

In some preferred embodiments, a dosing device in accordance with the present invention is configured such that the rotational axes of the dosing setting means and of the scale run or extend parallel to each other or are identical to increase the compactness of the scale. In some preferred embodiments, the rotational axes are configured such that they are identical or such that one rotational axis rotates about the other.

In some preferred embodiments, a particularly compact configuration of the dosing device can be achieved using a differential gear, e.g. a cam disc gear, a cycloid cam disc gear or so-called harmonic drive, etc. In such gears, the rotational movements run into each other, which enables increased compactness. A high reduction ratio can nonetheless be achieved, so as to make a translational movement of the scale superfluous. In some preferred embodiments, an annular cycle disc is provided, the rotational movement of which represents the second rotational movement. The circular inner circumference of the cycle disc is in contact with an eccentric which rotates with the first rotational movement and is coupled both to a setting ring and to the rotational movement of the dosing rod, such that a rotation of the setting ring with the first rotational movement causes a rotation of the dosing rod, likewise with the first rotational movement. Due to the contact between the eccentric and the inner circumference of the cycle disc, the cycle disc is rolled off via its cycloid outer toothing on a likewise cycloid inner toothing of a ring surrounding the cycle disc, wherein the number of cycloid teeth “Z1” of the cycloid disc and the number “Z2” of the cycloid inner teeth of the outer ring determine the reduction ratio i in accordance with the following formula:
i=(Z2−Z1)/Z2.

In some preferred embodiments, the scale of the dosing device is configured such that it does not undergo a translational movement along the longitudinal axis, but remains at a pre-set longitudinal position or axial position of the rotational axis of the scale or in a pre-set longitudinal section of the dosing device and/or remains without moving longitudinally when the second rotational movement is performed.

In some embodiments, the setting ring and the scale arc arranged concentrically, to achieve an even more compact structure for the dosing device. This also enables a priming button to he positioned at the rear end of the dosing device, i.e. the end opposite the outlet end of an injection apparatus. This priming button can then be positioned such that it acts on the stroke setting rod of the dosage setting means. In some embodiments, the priming button is arranged such that it is protected by the housing and only exposes its activating area. This can prevent the apparatus from being unintentionally primed, if, for example, the apparatus is dropped. The setting ring surrounds the scale and is connected or engaged directly or integrally to the first rotational axis of the first rotational movement. The connection runs or extends radially from the outside inwards towards the rotational axis, behind the scale. In this application, “behind” is used in the sense that the front end (distal end) of the injection apparatus serves to eject a fluid. Opposite this front end in the longitudinal direction or extent of the injection apparatus is the rear end (proximal end) to which a dosing device in accordance with the present invention is or can be attached.

In some embodiments, the setting ring is transparent or at least transparent at those locations at which it would hide the scale from a viewer, i.e. the setting ring is transparent at least in the longitudinal section of the dosing device in which the scale is also situated. In some embodiments, a magnifying lens can be provided in the transparent portion of the setting ring, to improve the legibility of the scale which, due to the reduction, can be formed with narrow broken lines or writing.

The present invention is also directed to an administering apparatus for administering a fluid product for medical, including veterinary, therapeutic, diagnostic, pharmaceutical, cosmetic and other applications or uses. Injection apparatus, including injection pens and inhalation apparatus, are some preferred examples of administering apparatus in accordance with the present invention.

Administering apparatus or devices in accordance with the present invention comprise a dosing device in accordance with the present invention, to administer substances in doses or selected amounts.

In some preferred embodiments, the axes of the first and/or second rotational movement coincide with the longitudinal axis of the administering apparatus or run or extend at least parallel to the longitudinal axis of the administering apparatus to achieve a compact structure.

### BRIEF DESCRIPTION OF THE DRAWINGS

FIG. 1a shows a cross-section through one embodiment of a dosing device in accordance with the present invention, and FIG. 1b shows a cross-section through a cycloid gear 40 used in the embodiment depicted in FIG. 1a.

FIG. 2 shows one embodiment of one end of an administering apparatus in accordance with the present invention.

### DETAILED DESCRIPTION

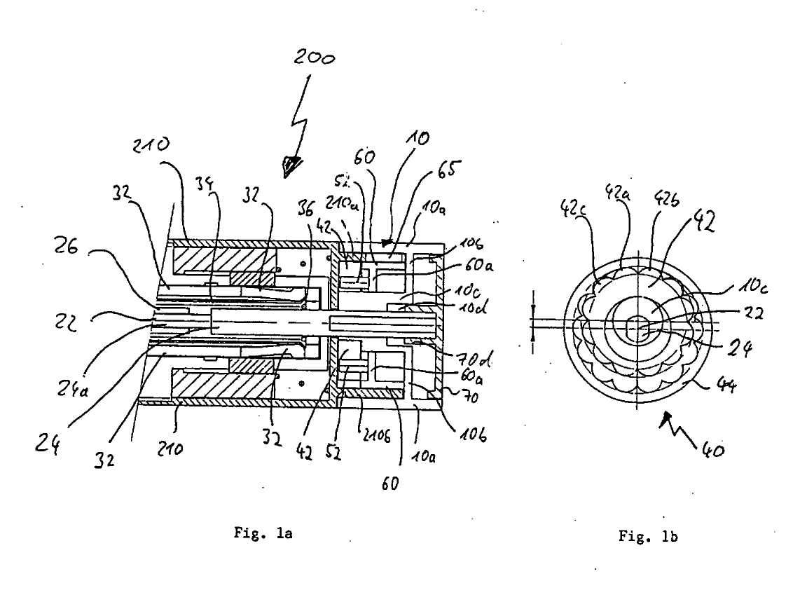

In one embodiment of the present invention, a dosage setting means 10 comprises a setting ring 10a. A bridge 10b protrudes from said setting ring 10a. This bridge 10b runs or extends radially inwardly, in some instances perpendicular to the setting ring, toward the longitudinal axis 22 of the apparatus 200. Before the bridge 10b reaches the longitudinal axis 22, it meets the eccentric sleeve 10c. This sleeve 10c rotates eccentrically about the axis 22 of the injection apparatus and the identical axis 22 of the dosing device 100 which occupies the section of the injection apparatus 200 shown on the right in FIG. 1a.

FIG. 1b shows a cross-section through a cycloid gear 40 used in the dosing device. The eccentric sleeve 10c surrounds the longitudinal axis 22 of an axial rod 24 which can be rotated about the longitudinal axis 22.

When the eccentric sleeve 10c is rotated, the axial rod 24 which is connected to the eccentric sleeve in a positive lock rotates with it. The right-hand end in FIG. 1a is the rear end which lies opposite the end at which the injection apparatus expels a product or fluid. The expelling end (not shown in FIG. 1a) is the front end.

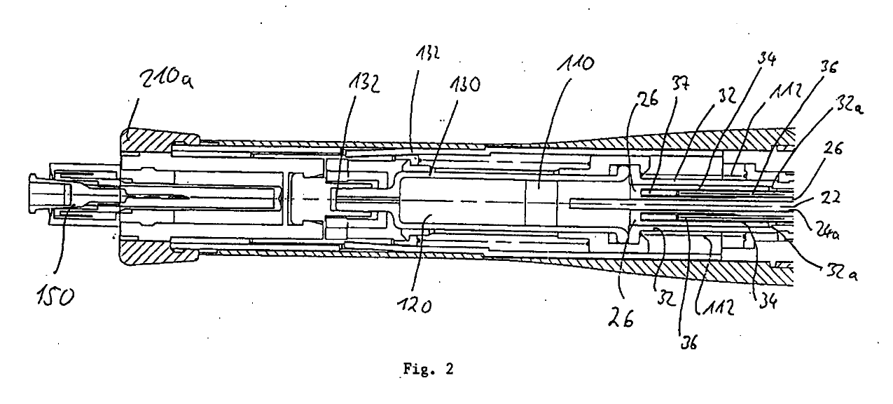

Towards the front end, the axial rod 24 transitions into a section 24a having a smaller diameter. This smaller section 24a comprises an outer thread which is in engagement with the inner thread of a stroke setting sleeve 26. The stroke setting sleeve is non-rotationally rounted, i.e. does not rotate with the axial rod. 24. When the axial rod 24 is rotated, the stroke setting sleeve 26 thus moves forward or backward. In this way, a stroke is set which acts on a piston 110 (see FIG. 2) when the injection apparatus is activated or triggered, the piston then pushing a fluid out of a reservoir 120 at the front end of the injection apparatus. The needle array 150 shown in FIG. 2 can be plugged or coupled on the front end 132 of the ampoule 130, once the needle array 150 has been turned. If the injection apparatus is then ready for operation, a latch (not shown) of an advancing sleeve 32 in the housing 210 can be released. This then causes an injection spring 112 to drive the advancing sleeve 32 forwards. The advancing sleeve 32 acts and the ampoule 130 and pushes it, together with the needle, out of the front end 210a of the housing 210 to perform the injection procedure.

The advancing sleeve 32 thereby slaves the stroke setting sleeve 26 via the edge 32a. Once a provided stopper 132 for the forward movement of the ampoule has been reached, a latch of an expelling sleeve 34 is released. This expelling sleeve 34 is driven in the forward direction by the spring 36. The expelling sleeve 34 is connected at its front end to a latching element 37. The latching element 37 is formed such that it slaves the stroke setting sleeve 26 in the forward direction when the expelling sleeve 34 is moved forwards. After travelling a predetermined distance, the expelling sleeve 34 latches into a latching location (not shown) provided in the advancing sleeve 32 and situated at a location corresponding to the predetermined distance. After the injection procedure, the latching element 37 thus travels a predefined distance in the longitudinal direction, causing delivery. The latching element 37 is formed such that, in the manner of a ratchet, it permits a movement of the stroke setting sleeve 26 relative to the latching element 37 when the latching element 37 is stationary and the stroke setting sleeve is moved in the forward direction by the dosing device, i.e. when the stroke setting sleeve 26 is moved forwards relative to the latching element 37. Should the latching element 37, however, want to move forwards relative to the stroke setting sleeve due to the spring action of the spring 36, then it slaves the stroke setting sleeve 26 due to the configuration of the latching.

In this way, the stroke setting sleeve 26 performs a stroke movement of equal length forwards, irrespective of its relative distance from the piston 110. The piston 110 is thus moved different distances forward in accordance with the longitudinal position of the stroke setting rod, such that a different delivery dosage is generated.

As described above, a stroke is thus set by means of the stroke setting sleeve 26, by rotating the setting ring 10a.

If the eccentric sleeve 10e is then rotated about the longitudinal axis 22, it not only slaves the axial rod 24 1:1, but also pushes—via the portion of the outer circumference which is furthest away from the longitudinal axis—against the inner circumference of a cycloid disc 42. In some preferred embodiments, this inner circumference is circular. Via its cycloid outer circumference, the cycloid disc rolls off an an inner circumference of a ring 44, which the eccentric sleeve 10c is rotated and thus moves along the inner circumference of the cycloid disc 42 with the eccentric point of the outer circumference. In this way, the protruding cycloid sections in the cycloid disc 42 are gradually pushed into correspondingly shaped cycloid indentations in the ring 44. In FIG. 1b, the cycloid sections 42a and 42b of the cycloid disc 42 have just fitted completely into a corresponding indentation in the ring 44. If the eccentric sleeve 10c in FIG. 1b is moved anti-clockwise, then the cycloid section 42c will be the next to roll into a corresponding indentation. This rolling-off movement of the cycloid disc causes a rotation of the cycloid disc, but one which is significantly slower than the rotation of the eccentric sleeve. The reduction ratio “i” is calculated to be i=(Z2−Z1)/Z2, wherein Z2 is the number of cycloid-like indentations in the ring 44 and Z1 is the number of matching cycloid sections in the cycloid disc 42.

The cycloid disc 42 is used in the dosing device in accordance with the present invention, as can be seen in FIG. 1a, to gyrate about the longitudinal axis 22 with the transmission ratio described above. In FIG. 1a, the cycloid disc 42 is situated at the upper end in contact with the housing section 210a of the housing 210 of the dosing device. In some embodiments, this housing 210 is also used as the housing for at least a portion of the injection apparatus. At the opposite, lower end of FIG. 1a, the cycloid disc 42 is spaced from the housing section 210b, i.e. the situation corresponds to that shown in FIG. 1b, in which the cycloid disc 42 is in its upper position and spaced at the bottom from the ring 44.

The outer circumference of the cycloid disc 42 in FIG. 1a thus comprises the cycloid cam sections. In the region of the longitudinal section of the cycloid disc 42, i.e. the region in which the cycloid disc rolls off, gyrating, an the inner circumference of the housing 210 (in particular at 210a and 210b), the inner circumference of the housing 210 comprises the corresponding cycloid-like recesses, i.e. is formed like the ring 44 already discussed in connection with FIG. 1b.

In some preferred embodiments, at least two cavities (not shown, but in which slaving pins 52 are situated) are situated in the cycloid disc 42 and fixedly connected to the scale 60. The scale 60 rotates about the longitudinal axis 22 without performing an eccentric movement. The trajectory covered by the rotational movement is pre-set by the inner wall of the housing 210, which corresponds to the ring 44. The scale 60 is formed as a cylindrical surface from which a stay 60a runs or extends radially inwardly and which is connected to the slaving pins 52. To view the scale, a cavity 65 or a window is provided in the casing, next to the section 210a, through which the scale can be read. The slaving pins 52 are fitted into the forementioned cavity in the cycloid disc with a clearance, such that they are slaved in the rotational movement of the cycloid disc but need not follow the eccentric rotational movement of the cycloid disc about the longitudinal axis 22. This generates a non-eccentric movement of the scale 60 about the longitudinal axis 22, which is slowed down as compared to the rotational movement of the axial rod 24 in accordance with the reduction ratio i.

Thus, in accordance with the present invention, a dosing device is achieved which allows a wide range of dosing adjustment, while the scale can be configured to be compact since a longitudinal movement of the scale to display a second dosage range, is not necessary.

In some embodiments, a priming button 70 is attached to the rear end of the injection apparatus and configured such that it can be pressed in along the longitudinal axis of the apparatus and toward the front end, to perform a priming strake which, for example, serves to evacuate the injection apparatus. To this end, a sleeve-shaped recess 10d is formed in the dosage setting means, in which a corresponding sleeve 70d of the priming button 70, running or extending symmetrically with respect to the longitudinal axis, is guided. This sleeve 70d surrounds the axial rod 24, wherein the priming button 70 concludes the axial rod 24 to the rear, so as to push the axial rod 24 forwards when the priming button 70 is moved forward to generate the priming stroke. In some embodiments, the priming button is also fitted into the setting ring or housing such that it does not protrude backward but can nonetheless be activated, for example by exposing at least a portion of its activating area.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A dosing device for an administering apparatus for administering a substance in doses, said dosing device comprising:
a) a dose selector for selecting a dose by a first rotational movement; and
h) a scale which can be rotated by a second rotational movement to display the selected dose; wherein
c) the first and second rotational movements are operably coupled such that the second rotational movement is slower then the first rotational movement.

2. The dosing device according to claim 1, further comprising a gear operably coupling the first and second rotational movements, wherein the gear is configured such that the rotational axes of the first and second rotational movement are one of parallel or coaxial.

3. The dosing device according to claim 2, wherein the gear comprises a differential gear.

4. The dosing device according to claim 3, wherein the differential gear comprises an eccentric which rotates with the first rotational movement and is formed as the drive sick of the differential gear.

5. The dosing device according to claim 4, wherein the differential gear is a cycloid cam disc gear.

6. The dosing device according to claim 4, wherein the gear comprises a driver side, the driven side being a cycloid cam disc, the rotational movement of which is coupled to the second rotational movement.

7. The dosing device according to claim 1, wherein the scale remains at a pre-set longitudinal position of the rotational axis of the second rotational movement at any rotation.

8. The dosing device according to claim 1, wherein the dose selector comprises a rotating setting ring for generating the first rotational movement, said setting ring being coupled to a rotary mechanism for setting the dose.

9. The dosing device according to claim 8, wherein the setting ring and the scale annularly surround the axis or axes of the first and second rotational movement.

10. The dosing device according to claim 8, wherein the setting ring comprises a transparent portion, and the scale and the transparent portion of the setting ring are arranged such that the scale can be viewed through the transparent portion.

11. An administering apparatus for administering a substance in doses, comprising a dosing device comprising:
a) a dose selector for selecting a dose by a first rotational movement; and
h) a scale which can be rotated by a second rotational movement to display the selected dose; wherein
c) the first and second rotational movements are operably coupled such that the second rotational movement is slower then the first rotational movement.

12. The administering apparatus according to claim 11, further comprising a gear operably coupling the first and second rotational movements.

13. The administering apparatus according to claim 12, wherein the gear comprises a cycloid cam disc gear.

14. The administering apparatus according to claim 12, wherein the apparatus has a longitudinal expansion direction, and wherein the axis of the first and/or second rotational movement is one of parallel or identical to the longitudinal expansion direction.

15. A dosing device for an administering apparatus for administering a substance in doses, comprising:
a) dosage setting means for setting a dosage of the substance to be administered by a first rotational movement; and
h) a scale which can be rotated by a second rotational movement for reading the dosage set; wherein
c) a gear couples the first rotational movement and the second rotational movement such that the second rotational movement is slower then the first rotational movement.

16. The dosing device according to claim 15, further comprising a gear operably coupling the first and second rotational movements, wherein the gear is configured such that the rotational axes of the first and second rotational movement are one of parallel or coaxial.

17. The dosing device according to claim 16, wherein the gear is a cycloid cam disc gear.

18. The dosing device according to claim 15, wherein the dosage setting means comprises a rotating setting ring for generating the first rotational movement, said setting ring being coupled to a rotary mechanism for setting the dose.

19. The dosing device according to claim 18, wherein the setting ring and the scale annularly surround the axis or axes of the first and second rotational movement.

20. The dosing device according to claim 19, wherein the setting ring comprises a transparent portion, and the scale and the transparent portion of the setting ring are arranged such that the scale can be viewed through the transparent portion.

21. An administering apparatus for administering a substance in doses, comprising a dosing device comprising:
a) dosage setting means for setting a dosage of the substance to be administered by a first rotational movement; and
h) a scale which can be rotated by a second rotational movement for reading the dosage set; wherein
c) a gear couples the first rotational movement and the second rotational movement such that the second rotational movement is slower then the first rotational movement.

22. The administering apparatus according to claim 21, wherein the apparatus has a longitudinal expansion direction, and wherein the axis of the first and/or second rotational movement is one of parallel or identical to the longitudinal expansion direction.
